(19) United States
(12) Patent Application Publication
(10) Pub. No.: US 2014/0004055 A1
(43) Pub. Date: Jan. 2, 2014

(54) SUNSCREEN COMPOSITIONS CONTAINING AN ULTRAVIOLET RADIATION-ABSORBING POLYESTER

(71) Applicants: SUSAN DALY, Basking Ridge, NJ (US); Rocco Vincent Burgo, Mullica Hill, NJ (US)

(72) Inventors: SUSAN DALY, Basking Ridge, NJ (US); Rocco Vincent Burgo, Mullica Hill, NJ (US)

(21) Appl. No.: 13/535,899

(22) Filed: Jun. 28, 2012

(51) Int. Cl. A61K 8/85; A61Q 17/04; A61K 8/06

(52) U.S. Cl. 424/59

(57) ABSTRACT

Compositions including a discontinuous oil phase substantially homogeneously dispersed in a continuous water phase, the oil phase including a sunscreen agent that includes a UV-absorbing polyester in an amount effective to provide the composition with an SPF of about 10 or greater and which is the polymerization reaction product of monomers including a UV-absorbing triazole, a diester, a diol and a tetrol polyol; and an oil-in water emulsifier component including an anionic oil-in-water emulsifier in an amount such that the composition includes about 0.3 percent or more by weight to about 3 percent by weight of the anionic oil-in-water emulsifier, where the composition is substantially free of a non-polymeric UV-absorbing sunscreen agent.

### FIELD OF THE INVENTION

The present invention relates to topically-acceptable sunscreen compositions comprising UV-absorbing polyesters.

### BACKGROUND OF THE INVENTION

The prolonged exposure to UV radiation, such as from the sun, can lead to the formation of light dermatoses and erythemas, as well as increase the risk of skin cancers, such as melanoma, and accelerate skin aging, such as loss of skin elasticity and wrinkling.

Numerous sunscreen compositions are commercially available with varying ability to shield the body from ultraviolet light. Unfortunately, many commercial sunscreens either sting or irritate the eye or skin. Accordingly, mild sunscreen formulations are desired by the consumer.

The challenge of creating mild sunscreens is further magnified if one imposes additional constraints on the sunscreen composition. For example, the inventors have recognized that it would be desirable to have mild, aesthetic sunscreen compositions that include a polymeric sunscreen compound (i.e., an ultraviolet radiation-absorbing polyester), and are substantially free of non-polymeric UV-absorbing sunscreen agents.

### SUMMARY OF THE INVENTION

Compositions of the present invention include a discontinuous oil phase that includes a sunscreen agent that includes a UV-absorbing polyester in an amount effective to provide the composition with an SPF of about 10 or greater. The discontinuous oil phase is substantially homogeneously distributed in a continuous water phase. The UV-absorbing polyester comprises the polymerization reaction product of monomers comprising a UV-absorbing triazole, a diester, a diol, and a tetrol polyol. The composition further comprises an oil-in-water emulsifier component comprising an anionic oil-in-water emulsifier in an amount of about 0.3 percent to about 3 percent by weight of said composition. The composition is substantially free of a non-polymeric UV-absorbing sunscreen agent.

### DETAILED DESCRIPTION OF THE INVENTION

Unless defined otherwise, all technical and scientific terms used herein have the same meaning as commonly understood by one of ordinary skill in the art to which the invention belongs. As used herein, unless otherwise indicated, all alkyl, alkenyl, and alkoxy groups may be straight, branched chain, or cyclic groups. As used herein, unless otherwise indicated, the term “molecular weight” refers to weight average molecular weight, (Mw).

Unless defined otherwise, all concentrations refer to concentrations by weight. Also, unless defined otherwise, the term “essentially free of,” with respect to a class of ingredients, refers to the particular ingredient(s) being present in a concentration less than is necessary for the particularly ingredient to be effective to provide the benefit or property for which it otherwise would be used, for example, less than about 1%, such as less than about 0.5%.

As used herein, “UV-absorbing” refers to a material or compound, e.g. a polymeric or non-polymeric sunscreen agent or a chemical moiety, which absorbs radiation in some portion of the ultraviolet spectrum (290 nm-400 nm), such as one having an extinction coefficient of at least about 1000 mol⁻¹ cm⁻¹, for at least one wavelength within the above-defined ultraviolet spectrum. SPF values disclosed and claimed herein are determined using the in-vitro method described herein below.

### UV-Absorbing Polyester

Embodiments of the invention relate to compositions including a sunscreen agent that comprises a UV-absorbing polyester. Such polyesters may be characterized as the polymerization reaction, e.g., esterification or transesterification, product of polyols, polyacids, polyanhydrides and/or polyesters. By “polyester,” it is meant a polymer having multiple repeat units, each of the repeat units including an ester functional group, [—COO—]. As such, the UV-absorbing polyester may include one or more “polyester backbone” portions, each polyester backbone portion having one or more ester functional groups that are derived by polymerization, as described herein. As used herein, “UV-absorbing polyester” may include residual free monomer which may be present resulting from the polymerization process.

According to certain embodiments, the UV-absorbing polyester is complex. By “complex,” it is meant that the UV-absorbing polyester includes terminal monofunctional compounds. The UV-absorbing polyester is fully or partially terminated (by reaction) with monofunctional acids, anhydrides, monofunctional alcohols, monofunctional epoxides and/or monofunctional esters.

According to certain embodiments, the UV-absorbing polyester is cross-linked. By “cross-linked” it is meant the UV-absorbing polyester has three or more terminal groups, each terminating a branch of the UV-absorbing polyester. Accordingly, the UV-absorbing polyester may be made using one or more polyfunctional monomers that has at least three total functional groups, for example four functional groups.

According to certain embodiments, the UV-absorbing polyester comprises a plurality of independent polyester moieties, each of which is terminated, or “capped”, by a UV-absorbing moiety. UV-absorbing polyesters that may be used in compositions according to the present invention are described in United States patent application publication number US2011/0104078 A1. In particular, UV-absorbing polyesters according to Scheme 6 of the application, and as further defined herein below, are useful in compositions of the present invention that are substantially free of non-polymeric UV-absorbing sunscreen agents.

The UV-absorbing polyester is UV-absorbing in that it includes UV-absorbing moieties, as discussed herein below, and therefore absorbs radiation in some portion of the ultraviolet spectrum (290 nm-400 nm), such as one having an extinction coefficient of about 1000 mol⁻¹ cm⁻¹ or more, for example greater than 10,000 or 100,000 or 1,000,000 mol⁻¹ cm⁻¹, for at least one wavelength within the above-defined ultraviolet spectrum. The UV-absorbing moiety may absorb predominantly in the UV-A portion (320 nm to 400 nm) or predominantly in the UV-B portion (290 nm to 320 nm) of the ultraviolet spectrum. Particularly suitable examples UV-absorbing moieties include UV-absorbing triazoles. By “UV-absorbing triazole” it is meant a UV-absorbing moiety containing a five-membered heterocyclic ring with two carbon and three nitrogen atoms. Typical UV-absorbing triazoles are benzotriazoles, which include the mentioned five-membered heterocyclic ring fused with a six-membered homocyclic aromatic ring. Examples of UV-absorbing triazoles include, for example, compounds of the formula (II) or (III):

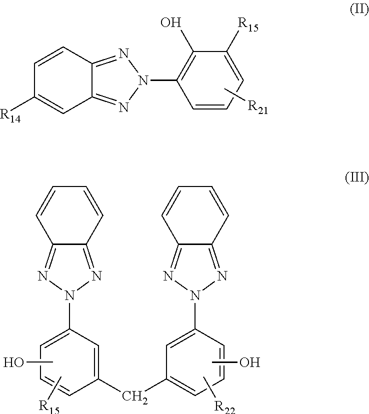

wherein R₁₄ is an optional C₁-C₁₈ alkyl or hydrogen; R₁₅ and R₂₂, independently, are optionally C₁-C₁₈ alkyl that may be substituted with a phenyl group, and R₂₁ is an optional functional group such as a C₁-C₈ alkyl that may include an ester linkage containing a methyl group.

The polyester moieties may each include or consist of n repeat units such as (IV) or (V) below:

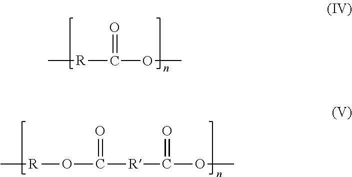

In structures (IV) and (V): R and R′ represent hydrocarbons such as alkyl, aryl, or aralkyl chains (saturated or unsaturated) having a carbon chain length ranging independently from C₁-C₁₀₀, such as C₄-C₅₀, such as C₆-C₄₀; n is the degree of polymerization of each of the independent polyester moieties and may range from 1 to about 20, such as from 1 to about 10, such as from 1 to about 5. The total degree of polymerization, i.e., the sum of n for all polyester moieties in the UV-absorbing polyester, may range from 4 to about 25, such as from about 5 to about 20, such as from 5 to about 10.

According to certain embodiments, the UV-absorbing polyester has a weight average molecular weight (Mw) of about 2,000 or more, such as about 4,000 or more, such as from about 4,000 to about 4,500, as determined by gel permeation chromatography using, for example, the following conditions and detection system.

Determination of Mw may be performed using the following gel permeation chromatography (GPC) method and equipment. A suitable liquid chromatography system is an Agilent 1100/1200 Series high performance liquid chromatography system, the hardware of the which includes 5 modules; a G1379A degasser, a Model G1310A isocratic pump, a 1110 automatic liquid sampler Model G1313A, a Model G1316A thermostatted column compartment, and a Model G1362A refractive index detector (RID). The system is controlled using Agilent LC Chemstation software, Revision B.03.02. The system is fitted with two Varian MesoPore GPC Columns, 300×7.5 mm, 3 um, multipore. The samples are dissolved in ACS HPLC grade tetrahydrofuran (THF) to a concentration of approximately 1.0 mg/ml. The THF contains 250 ppm butylated hydroxytoluene (BHT) as oxidation inhibitor. The THF is filtered using 0.45 um Millipore filter before being used as the mobile phase solvent and sissolution solvent. The solvent is degassed continuously by the vacuum degasser in the system. The mobile phase flow rate is 1 mL/min. The two column set is held at 45° C. in the column compartment. The injection volume is 200 microliters. The run time is thirty minutes.

Calibration of the GPC column is performed using 10 narrow molecular weight distribution polystyrene standards with molecular weights of 162, 580, 1110, 1530, 2340, 3790, 5120, 7210, 12830, and 19640 Daltons, respectively. The standards may be purchased from Agilent-Varian. Each standard is injected and the molar mass is linearly regressed against elution volume to give the calibration line. Molecular weight calculations for the polyesters are determined using Agilent GPC Addon Revision B.01.01, an add-on to Agilent Chemstation software. All results for inventive polyesters given in the units of Daltons are relative to the polystyrene standards.

In certain embodiments, in order to enhance water-resistance and spreadability, the UV-absorbing polyester may have a low water-solubility. By “water-solubility” it is meant the maximum weight percentage of polyester (relative to polyester plus water) that can be placed into 100 grams deionized water and agitated so that a clear solution is obtained and remains visually homogeneous and transparent at ambient temperature for 24 hours. For example, in certain embodiments, the UV-absorbing polyester may have a water-solubility that is about 3% or less, such as about 1% or less.

The UV-absorbing polyesters suitable for use in compositions of the present invention may be synthesized by various means known to those skilled in the art, e.g., ring opening of a lactone (cyclic ester) that bears a UV-absorbing moiety; a condensation reaction of a UV-absorbing monomer having both acid and alcohol functionality (e.g., an “A-B” condensation reaction); condensing a polyol functional monomer and a polyacid functional monomer, one or both of which includes UV-absorbing moieties; and the like.

One particularly suitable process for making the UV-absorbing polyester is via a transesterification reaction, such as by reacting a polyfunctional hydroxyl, e.g., a tetrol polyol (a molecule having four alcohol functional groups), a diol, a di-carboxylic acid, and an ester-functional UV-absorbing monomer. For example, three monomers, each absent a UV-absorbing moiety, e.g., a diol, a tetrol polyol and a di-carboxylic acid, may be reacted with a fourth monomer, e.g., a UV-absorbing triazole having an ester functionality, to produce a UV-absorbing polyester. The mole ratio of monomers may be selected such that the ratio of various monomer pairs is from about 0.25:1 to about 4:1. According to one embodiment, the mole fraction of UV-absorbing monomer, e.g., UV-absorbing triazole, relative to the total number of moles of all monomers used in the reaction (including the UV-absorbing monomer) is selected to be about 0.39 to about 0.60, or about 0.37 to about 0.42. According to another embodiment, this mole fraction is selected to be about 0.45 or less.

One particularly suitable UV-absorbing polyester is formed by a transesterification reaction of the following monomers: (1) dimerdiol, C₃₆H₇₂₀, CAS No. 147853-32-5, which is a C₃₆ diol; (2) di-trimethylolpropane, C₁₂H₂₆O₅, CAS No. 23235-61-2, which is a tetrafunctional alcohol (tetrol polyol) derived from the dimerization of trimethylolpropane; (3) dimethyladipate, C₈H₁₄O₄, CAS No 627-93-0, the methyl ester of adipic acid; and (4) benzenepropanoic acid, 3-(2h-benzotriazol-2-yl)-5-(1,1-dimethylethyl)-4-hydroxy-, methylester, C₂₀H₂₃N₃O₃, CAS No 84268-33-7, a UV-absorbing monomer (includes a UV-absorbing triazole). Dimerdiols are described in United States patent, U.S. Pat. No. 7,427,640.

According to certain embodiments, the sunscreen agent consists of, or consists essentially of, the UV-absorbing polyester, as defined herein. According to certain other embodiments, the sunscreen agent may include additional UV-absorbing polymers, other than those UV-absorbing polyesters, as defined herein, and/or non-UV-absorbing, light-scattering particles. Additional UV-absorbing polymers are molecules that can be represented as having one or more structural units that repeat periodically, e.g., at least twice, to generate the molecule, and may be UV-absorbing polyesters, other than those as defined and claimed in this specification.

Additional UV-absorbing polymers may have a molecular weight of greater than about 1500. Examples of suitable additional UV-absorbing polymers include benzylidene malonate silicone, including those described in U.S. Pat. No. 6,193,959, to Bernasconi et al. A particularly suitable benzylidene malonate includes “Parsol SLX,” commercially available from DSM (Royal DSM N.V.) of Heerlen, Netherlands. Other suitable additional UV-absorbing polymers are disclosed in U.S. Pat. No. 6,962,692; U.S. Pat. No. 6,899,866; and/or U.S. Pat. No. 6,800,274; including hexanedioic acid, polymer with 2,2-dimethyl-1,3-propanediol, 3-[(2-cyano-1-oxo-3,3-diphenyl-2-propenyl)oxy]-2,2-dimethylpropyl 2-octyldodecyl ester; sold under the trade name “POLYCRYLENE,” commercially available from the HallStar Company of Chicago, Ill. When utilized, such additional UV-absorbing polymers may be used at concentrations of about 1% or more, for example about 3% or more.

Non-UV-absorbing, light-scattering particles do not absorb in the UV spectrum, but may enhance SPF by scattering of the incident UV radiation. Examples of non-UV-absorbing, light-scattering particles include solid particles having a dimension, e.g., average diameter, from about 0.1 micron to about 10 microns. In certain embodiments, the non-UV-absorbing, light-scattering particle is a hollow particle comprising, or consisting essentially of, an organic polymer or a glass. Suitable organic polymers include acrylic polymers, including acrylic/styrene copolymers, such as those known as SUNSPHERES, which are commercially available from Dow Chemical of Midland, Mich. Suitable glasses include borosilicate glasses such as those described in published United States Patent Application US20050036961A1, entitled, “AESTHETICALLY AND SPF IMPROVED UV-SUNSCREENS COMPRISING GLASS MICROSPHERES”.

### Topical Composition

In one embodiment, a composition suitable for topical/cosmetic use for application to the human body, e.g., keratinaceous surfaces such as the skin, hair, lips, or nails, and especially the skin, is provided. The composition includes one or more UV-absorbing polyesters described herein. As discussed above, the concentration of the UV-absorbing polyester is sufficient to provide an SPF of about 10 or greater, particularly in the absence or substantial absence of other UV-absorbing polymers or non-polymeric UV-absorbing sunscreen agents as described herein. Accordingly, the concentration of the UV-absorbing polyester may vary from about 5% to about 50%, such as from about 7% to about 40%, such as from about 10% to about 25% of the composition. In certain embodiments, the concentration of UV-absorbing polyester is about 10% or more, such as about 15% or more, such about 25% or more of the composition. According to certain embodiments where the sunscreen agent consists essentially of the UV-absorbing polyester, the concentration of the UV-absorbing polyester may be about 15% or more.

The concentration of non-UV-absorbing sunscreen agents, if present, may be about 1% or more, such as from about 1% to about 10%, such as from about 2% to about 5%. In certain embodiments where the UV-sunscreen agent further includes a non-UV-absorbing sunscreen agent in amounts as discussed above, compositions of the present invention may have an SPF of about 20 or greater.

Compositions of the present invention are substantially free of non-polymeric UV-absorbing sunscreen agents. By “substantially free of non-polymeric UV-absorbing sunscreen agents,” it is meant that the compositions do not contain non-polymeric UV-absorbing sunscreen agents in an amount effective to provide the compositions with an SPF of greater than 2 in the absence of the UV-absorbing polyesters, as determined via the in vitro method described herein below. For example, the compositions of the invention will contain about 1% or less, or about 0.5% or less, of such non-polymeric UV-absorbing sunscreen agents. One example of non-polymeric UV-absorbing sunscreen agents that the composition is substantially free of typically may be characterized as “organic” (include predominantly or only atoms selected from carbon, hydrogen, oxygen, and nitrogen) and having no definable repeat unit and typically having molecular weights that are about 600 daltons or less, such as about 500 daltons or less, such as less than 400 daltons. Examples of such compounds, sometimes referred to as “monomeric, organic UV-absorbers” include, but are not limited to: methoxycinnamate derivatives such as octyl methoxycinnamate and isoamyl methoxycinnamate; camphor derivatives such as 4-methyl benzylidene camphor, camphor benzalkonium methosulfate, and terephthalylidene dicamphor sulfonic acid; salicylate derivatives such as octyl salicylate, trolamine salicylate, and homosalate; sulfonic acid derivatives such as phenylbenzimidazole sulfonic acid; benzone derivatives such as dioxybenzone, sulisobenzone, and oxybenzone; benzoic acid derivatives such as aminobenzoic acid and octyldimethyl para-amino benzoic acid; octocrylene and other β,β-diphenylacrylates; dioctyl butamido triazone; octyl triazone; butyl methoxydibenzoyl methane; drometrizole trisiloxane; and menthyl anthranilate.

Other non-polymeric UV-absorbing sunscreen agents that the composition is substantially free of may include ultraviolet-absorbing particles, such as certain inorganic oxides, including titanium dioxide, zinc oxide, and certain other transition metal oxides. Such ultraviolet screening particles are typically solid particles having a diameter from about 0.1 micron to about 10 microns.

The compositions of the present invention may be used for a variety of cosmetic uses, especially for protection of the skin from UV radiation. The compositions, thus, may be made into a wide variety of delivery forms. These forms include, but are not limited to, suspensions, dispersions, solutions, or coatings on water soluble or water-insoluble substrates (e.g., substrates such as organic or inorganic powders, fibers, or films). Suitable product forms include lotions, creams, gels, sticks, sprays, ointments, mousses, and compacts/powders. The composition may be employed for various end-uses, such as recreation or daily-use sunscreens, moisturizers, cosmetics/make-up, cleansers/toners, anti-aging products, or combinations thereof. The compositions of the present invention may be prepared using methodology that is well known by an artisan of ordinary skill in the field of cosmetics formulation.

Compositions of the present invention include a continuous water phase in which a discontinuous oil phase that includes the UV-absorbing polyester is substantially homogeneously distributed. In certain embodiments, the UV-absorbing polyester is dissolved, as opposed to being dispersed or suspended, within the oil phase. The oil phase may, in turn, be stabilized within the water phase. The oil phase may be such that it is present in discrete droplets or units having an average diameter of about one micron to about 1000 microns, such as from about 1 micron to about 100 microns.

The relative concentrations of water phase and oil phase may be varied. In certain embodiments the percentage by weight of water phase is from about 10% to about 90%, such as from about 40% to about 80%, such as from 50% to about 80%; wherein the balance is oil phase.

The percentage of water included in the compositions may range from about 20% to about 90%, such as from about 20% to about 80%, such as from about 30% to about 70%, such as from about 51% to about 80%, such as from about 51% to about 70%, such as from about 51% to about 60%.

### Topical Carrier

The one or more UV-absorbing polymers in the composition may be combined with a “cosmetically-acceptable topical carrier,” i.e., a carrier for topical use that is capable of having the other ingredients dispersed or dissolved therein, and possessing acceptable properties rendering it safe to use topically. As such, the composition may further include any of various functional ingredients known in the field of cosmetic chemistry, for example, emollients (including oils and waxes) as well as other ingredients commonly used in personal care compositions, such as humectants, thickeners, opacifiers, fragrances, dyes, solvents for the UV-absorbing polyester, among other functional ingredients. Suitable examples of solvents for the UV-absorbing polyester include dicaprylyl carbonate available as CETIOL CC from Cognis Corporation of Ambler, Pa. In order to provide pleasant aesthetics, in certain embodiments of the invention, the composition is substantially free of volatile solvents, and, in particular, C₁-C₄ alcohols such as ethanol and isopropanol.

Furthermore, the composition may be essentially free of ingredients that would render the composition unsuitable for topical use. As such, the composition may be essentially free of solvents such as volatile solvents, and, in particular, free of volatile organic solvents such as ketones, xylene, toluene, and the like.

### Emulsifiers

The inventors surprisingly have found that UV-protective, mild sunscreens that are substantially free of non-polymeric UV-absorbing sunscreen agents can be made by forming an oil-in-water (O/W) emulsion comprising a UV-absorbing polyester and particular emulsifiers in a particular weight range. As such, compositions of the present invention include an O/W emulsifier component that includes one or more O/W emulsifiers. By “O/W emulsifier,” it is meant any of a variety of molecules that are suitable for emulsifying discrete oil-phase droplets in a continuous water phase. By “low molecular weight emulsifiers,” it is meant emulsifiers having a molecular weight of about 2000 daltons or less, such as about 1000 daltons or less. The O/W emulsifier may be capable of lowering the surface tension of pure deionized water to 45 dynes per centimeter when added to pure deionized water at a concentration of O/W emulsifier of 0.5% or less at room temperature. O/W emulsifiers are sometimes characterized as having a hydrophile-lipophile balance (HLB) that is about 8 or more, such as about 10 or more.

The O/W emulsifier component comprises one or more anionic emulsifiers, such that the total concentration of anionic emulsifier in the composition is about 3% or less. Examples of suitable chemical classes of anionic emulsifiers are alkyl, aryl or alkylaryl, or acyl-modified versions of the following moieties: sulfates, ether sulfates, monoglyceryl ether sulfates, sulfonates, sulfosuccinates, ether sulfosuccinates, sulfosuccinamates, amidosulfosuccinates, carboxylates, amidoethercarboxylates, succinates, sarcosinates, amino acids, taurates, sulfoacetates, and phosphates. Notable anionic emulsifiers are phosphate esters, such as cetyl phosphate salts, such as potassium cetyl phosphate. In certain embodiments, the concentration of the one or more anionic emulsifiers is from about 0.3% to about 3%, such as from about 1% to about 3%, such as from about 0.5% to about 2.5%, of the weight of the composition. According to certain embodiments, the O/W emulsifier component consists essentially of the one or more anionic emulsifiers.

According to certain embodiments, the O/W emulsifier component is essentially free of non-ionic emulsifiers having an alcohol-functional group with a hydrocarbon chain length of 14-22 carbon atoms. Chemical classes of non-ionic emulsifiers having an alcohol functional group may include fatty alcohols, such as various saturated or unsaturated, linear or branched, C₇-C₂₂ unethoxylated, aliphatic alcohols, such as those having a single —OH group. The fatty alcohol may be derived from plant or animal oils and fats having at least one pendant hydrocarbon-comprising chain. The fatty alcohol may have from 14 to about 22 carbon atoms, such as from about 16 to about 18 carbon atoms. Examples of unbranched fatty alcohols include cetyl alcohol and stearyl alcohol.

According to certain other embodiments, the O/W emulsifier component is essentially free of cationic emulsifiers, such as alkyl quaternaries, benzyl quaternaries, ester quaternaries, ethoxylated quaternaries, and alkyl amines.

According to certain embodiments, in addition to the anonic oil-in-water emulsifier(s) discussed above, the O/W emulsifier component includes an additional emulsifier such as a non-ionic emulsifier that is devoid of alcohol functional groups, an amphoteric emulsifier, and/or a polymeric emulsifier. Examples of suitable chemical classes of non-ionic emulsifier include ethoxylates of amides; polyoxyethylene derivatives of polyol esters; noncrosslinked silicone copolymers such as alkoxy or alkyl dimethicone copolyols, silicones having pendant hydrophilic moieties such as linear silicones having pendant polyether groups or polyglycerin groups; and crosslinked elastomeric solid organopolysiloxanes comprising at least one hydrophilic moiety.

Examples of suitable chemical classes of amphoteric emulsifiers include alkyl betaines, amidoalkyl betaines, alkylamphoacetates; amidoalkyl sultaines; amphophosphates; phosphorylated imidazolines; carboxyalkyl alkyl polyamines; alkylimino-dipropionates; alkylamphoglycinates (mono or di); alkylamphoproprionates; N-alkyl β-aminoproprionic acids; and alkylpolyamino carboxylates. Examples of suitable chemical classes of polymeric emulsifier include copolymers based on acrylamidoalkyl sulfonic acid such as Aristoflex® AVC and Aristoflex® HMB by Clariant Corporation; and Granthix APP by Grant Industries, Inc.

### Film-Forming Polymers

In certain embodiments of the invention, compositions of the present invention include a film forming polymer. By “film-forming polymer,” it is meant a polymer that when dissolved, emulsified, or dispersed in one or more diluents, permits a continuous or semi-continuous film to be formed when it is spread with a liquid vehicle onto smooth glass, and the liquid vehicle is allowed to evaporate. As such, the polymer should dry on the glass in a manner in which over the area which it is spread should be predominantly continuous, rather than forming a plurality of discrete, island-like structures. Generally, the films formed by applying compositions on the skin according to embodiments of the invention described herein, are less than, on average, about 100 microns in thickness, such as less than about 50 microns.

In contrast to polymeric UV-absorbing polymers, film-forming polymers generally do not absorb ultraviolet radiation and therefore do not meet the requirements for UV-absorbing polymers. Film-forming polymers may be useful in compositions of the present invention in that they may enhance the UV-protection (UV-A, UV-B or both) of the composition and/or enhance the waterproofing or water resistance of the composition.

Suitable film-forming polymers include natural polymers such as polysaccharides or proteins and synthetic polymers such as polyesters, polyacrylics, polyurethanes, vinyl polymers, polysulfonates, polyureas, polyoxazolines, and the like. Specific examples of film-forming polymers include, for example, hydrogenated dimer dilinoleyl/dimethylcarbonate copolymer, available from Cognis Corporation of Ambler, Pa. as COSMEDIA DC; copolymer of vinylpyrrolidone and a long-chain α-olefin, such as those commercially available from ISP Specialty Chemicals of Wayne, N.J. as GANEX V220; vinylpyrrolidone/tricontanyl copolymers available as GANEX WP660 also from ISP; water-dispersible polyesters, including sulfopolyesters such those commercially available from Eastman Chemical as EASTMAN AQ 38S. The amount of film-forming polymer present in the composition may be from about 0.1% to about 5%, or from about 0.1% to about 3%, or from about 0.1% to about 2%.

In certain embodiments, the composition includes an emollient used for the prevention or relief of dryness and for the protection of the skin, as well as solubilizing the UV-absorbing polyester. Suitable emollients include mineral oils, petrolatum, vegetable oils (e.g. triglycerides such as caprylic/capric triglyceride), waxes and other mixtures of fatty esters, including but not limited to esters of glycerol (e.g, isopropyl palmitate, isopropyl myristate), and silicone oils such as dimethicone. In certain embodiments, mixtures of triglycerides (e.g. caprylic/capric triclycerides) and esters of glycols (e.g. isopropyl myristate) may be used to solubilize the UV-absorbing polyesters.

In certain embodiments, the composition includes a pigment suitable for providing color or hiding power. The pigment may be one suitable for use in a color cosmetic product, including compositions for application to the hair, nails and/or skin, especially the face, Color cosmetic compositions include, but are not limited to, foundations, concealers, primers, blush, mascara, eyeshadow, eyeliner, lipstick, nail polish and tinted moisturizers.

The pigment suitable for providing color or hiding power may be composed of iron oxides, including red and yellow iron oxides, titanium dioxide, ultramarine and chromium or chromium hydroxide colors, and mixtures thereof. The pigment may be a lake pigment, e.g., an organic dye such as azo, indigoid, triphenylmethane, anthraquinone, and xanthine dyes that are designated as D&C and FD&C blues, browns, greens, oranges, reds, yellows, etc., precipitated onto inert binders such as insoluble salts. Examples of lake pigments include Red #6, Red #7, Yellow #5 and Blue #1. The pigment may be an interference pigment. Examples of interference pigments include those containing mica substrates, bismuth oxycloride substrates, and silica substrates, for instance mica/bismuth oxychloride/iron oxide pigments commercially available as CHROMALITE pigments (BASF), titanium dioxide and/or iron oxides coated onto mica such as commercially available FLAMENCO pigments (BASF), mica/titanium dioxide/iron oxide pigments including commercially available KTZ pigments (Kobo products), CELLINI pearl pigments (BASF), and borosilicate-containing pigments such as REFLECKS pigments (BASF).

The compositions of the present invention may further comprise one or more other cosmetically active agent(s). A “cosmetically active agent” is a compound that has a cosmetic or therapeutic effect on the skin, e.g., agents to treat wrinkles, acne, or to lighten the skin. The cosmetically active agent will typically be present in the composition of the invention in an amount of from about 0.001% to about 20% by weight of the composition, e.g., about 0.01% to about 10% such as about 0.1% to about 5% by weight of the composition.

In certain embodiments the composition has a pH that is from about 4.0 to about 8.0, such as from about 5.5 to about 7.0.

Compositions of the present invention have low irritation tendencies. Irritation may be measured using, for example, the MODIFIED TEP TEST as set forth below. A lower MODIFIED TEP value of a composition tends to indicate less irritation associated therewith, as compared to a composition having a higher MODIFIED TEP value, which composition tends to cause higher levels of irritation.

Applicants have recognized that compositions of the present invention have surprisingly low MODIFIED TEP values/lower irritation associated therewith. For example, in certain embodiments, the compositions have a MODIFIED TEP value, as determined according to the MODIFIED TEP TEST as set forth below, of about 0.45 or less. In certain other embodiments, the compositions exhibit a MODIFIED TEP value of about 0.40 or less, such as about 0.35 or less, such as about 0.30 or less.

The compositions of the present invention may be prepared using mixing and blending methodology that is well known by an artisan of ordinary skill. In one embodiment of the invention, a method of making a composition of the present invention includes preparing an oil phase by mixing at least the UV-absorbing polyester with optional oil-soluble or oil-miscible ingredients; and preparing a water phase, by mixing water and optional water-soluble or water-miscible ingredients. The oil phase and the water phase may then be mixed in a manner sufficient to substantially homogeneously disperse the oil phase in the water phase such that the water phase is continuous and the oil phase discontinuous.

The compositions of the present invention can be used by topically administering to a mammal, e.g., by the direct laying on, wiping or spreading of the composition on the skin or hair of a human.

The following MODIFIED TEP TEST is used in the instant methods and in the following Examples. In particular, as described above, the MODIFIED TEP TEST is used to determine when a composition has reduced irritation according to the present invention.

### Modified TEP Test:

The MODIFIED TEP TEST is designed to evaluate the ability of a test material to disrupt the permeability barrier formed by a confluent monolayer of Madin-Darby canine kidney (MDCK) cells. MDCK cells grown to confluence on porous filters are used to assess trans-epithelial permeability, as determined by the leakage of fluorescein dye through the monolayer. The MDCK permeability barrier is a model for the outermost layers of the corneal epithelium and this system can therefore be considered to reflect early changes in the development of eye irritation in vivo.

The following equipment is suitable for the MODIFIED TEP TEST: Packard Multiprobe 104 Liquid handling system; BioTek Washer, model number ELx405; and BioTek Powerwave XS microplate reader with a 490 nm filter. Disposable lab ware includes: Corning Support Transwell 24-well cell culture plate with microporous membrane, Cat. No. 29445-100 or 29444-580, MFG. No. 3397; Corning Receiver 24-well Tissue Culture Plate, Cat No. 29444-100, MFG. No. 3527; disposable 200 μL tips Cat. No. 82003-196; Eppendorf 5 mL combitips plus Cat No. 21516-152; Sodium Chloride 0.9% (w/v) Aqueous Cat. No. RC72105; and sterile 15 mL polypropylene centrifuge tubes. Reagents supplied by Life Technologies include: Hank's Balanced Salt Solution (10×) without Phenol Red Cat. No. 14065056 and Sodium Bicarbonate Solution, 7.5% Cat No. 25080094, Minimum Essential Medium (MEM) (1×), Cat No. 11095072, Fetal Bovine Serum, HI Cat No. 10082147, Antibiotic Antimycotic 100× Cat No. 15240096, L-Glutamine 200 mM (100×) Cat No. 25030081, Sodium Fluorescein, Sigma Cat. No. F-6377 is provided by Sigma/Aldrich.

A cell line, ATCC CCL 34 MDCK (NBL-2) (Kidney: Canis familiaris), is maintained in accordance ATCC (Manassas, Va.) recommendations. Cell cultures are harvested by trypsinization and seeded into Support Transwell24 plates containing complete MEM, 48 hours prior to testing at a concentration of 5×10⁵ cells per mL. Reagents are prepared: (1) 1×HBSS Buffer by combining 200 mL Hank's Balanced Salt Solution (HBSS) (10×) without phenol red with 9.3 mL Sodium Bicarbonate and increasing the volume to 2000 mL with distilled water. The pH should be in the range of 6.8-7.2 and the solution should be warmed to 37 C; (2) a 200 ug/mL stock solution of sodium fluorescein in HBSS Buffer; (3) Complete Minimum Essential Medium (MEM) is prepared by combining 100 mL's of Fetal Bovine Serum, 10 mL's of Antibiotic Antimycotic 100×, and 10 mL's of L-Glutamine 200 mM (100×) to 1000 mL's of MEM (1×).

Permeability of the membrane is confirmed by including a No Cell Control that is run with each day of testing. Sunscreen test compositions are evaluated full strength.

Inserts are washed to remove cell medium. A 24-well cell culture plate, Corning Cat No. 29445-100, containing a confluent monolayer of MDCK cells is removed from the incubator. Each 24-well cell culture plate includes an insert which holds an inner well with a microporous membrane cell growth surface suspended into a lower well. The insert containing the cell cultures is washed 5× (BioTek Washer) with warm HBSS to remove culture medium and serum. The bottom portion of the 24-well cell culture plate is washed with warm HBSS 3× and on the last wash 1 mL of HBSS is dispensed in each bottom well.

Four wells in the 24-well plate are used per sunscreen test composition, so a single 24-well plate can be used to test up to 6 sunscreen test compositions. The sunscreen test compositions are added directly to the insert well, Neat (100%), 200 μL per insert well. The 24-well cell culture plate is then returned to the incubator for a 1 hour incubation period.

Upon completion of the first incubation step, the 24-well cell culture plate is removed from the incubator and washed manually to remove test composition. Approximately 200 μL of HBSS is added to each inner well and allowed to soak for approximately 1 minute. The test composition and HBSS are then decanted from the individual wells. Any residual sample is removed by delicately flooding the inserts with HBSS and decanting. When the insert is free of residual test composition, a 10× wash (Bio Tek Washer) with warm HBSS should be done. The bottom wells are washed with warm HBSS 3× and on the last wash 1 mL of HBSS (receiver buffer) is dispensed into each bottom well.

The insert is placed back into the bottom plate containing 1 mL HBSS (receiver buffer), sodium fluorescein is added to each inner well, 2004 per well, and the plate is returned to the incubator for a period of 45 minutes.

After the 45 minute incubation, the sodium fluorescein containing first plate is removed from the incubator, the upper insert is removed, and the amount of dye that has leaked into the receiver buffer in the lower well is determined by the Powerwave XS microplate reader. The fluorescence is read spectrophotometrically at 490 nm. Data is printed and recorded.

The insert is then placed into an empty, temporary, 24 well bottom plate on the Bio Tek Washer for a 10×HBSS wash. Care is taken to ensure that the sodium fluorescein has been washed off and there is no residual fluorescein in the top (inner) or bottom wells.

The washed insert is placed into a fresh 24-well receiver cell culture plate, Corning Cat No. 29445-100. Both the inner wells of the insert and the bottom plate receive complete minimum essential medium (MEM, Life Technologies, Cat No. 11095072. Approximately 1 mL of complete MEM is added to the bottom wells and 200 μL is added to the inner wells. The 24-well cell culture plate is then incubated for 3 hours.

After the 3 hour incubation the 24-well cell culture plate is removed from the incubator. The insert containing the cell cultures is washed 5× (BioTek Washer) with warm HBSS to remove culture medium and serum. The bottom plate is washed with warm HBSS 3× and on the last wash 1 mL of HBSS is dispensed in each bottom well (receiver buffer).

Sodium fluorescein is added to each inner insert well, 2004 per well, and the plate is reassembled and returned to the incubator for a period of 45 minutes.

After the 45 minute incubation, the sodium fluorescein containing plate is removed from the incubator, the insert is removed and discarded, and the amount of dye that has leaked into the lower well is determined by the Powerwave XS microplate reader. The fluorescence is read spectrophotometrically at 490 nm. Data is printed and recorded.

The spectrophotometric measurement (fluorescein leakage) value for each of the four repeats of a given sunscreen test composition is used to calculate an average fluorescein leakage value for the sunscreen test composition. The average fluorescein leakage value of the four “no cell control” wells is also calculated. The Modified TEP Score is calculated by dividing the average fluorescein leakage value of the sunscreen test composition by that of the no cell control.

Additional details of the TEP test are described in the following publication: Tchao, R. (1988) Trans-epithelial Permeability of Fluorescein In Vitro as an Assay to Determine Eye Irritants. Alternative Methods in Toxicology 6, Progress in In Vitro Toxicology (ed. A. M. Goldberg), 271.

### EXAMPLES

The following examples illustrate the preparation and efficacy of compositions of the present invention.

### Example 1

The following example illustrates the low irritation of certain compositions of the present invention. Inventive compositions (E1-E2) include UV-absorbing polyesters, are substantially free of non-polymeric UV-absorbing sunscreen agents and were prepared as shown in Table 1 and described below.

TABLE 1

| | C1 | C2 | E1 | E2 |
|---|---|---|---|---|
| Water | 59.4 | 61.4 | 63.4 | 64.8 |
| Amigel | 0.3 | 0.3 | 0.3 | 0.3 |
| phenonip XB | 1 | 1 | 1 | 1 |
| Pemulen TR-2 | 0.3 | 0.3 | 0.3 | 0.3 |
| UV-Absorbing Polyester A | 15 | 15 | 15 | 15 |
| (80% solution in dicaprylyl | | | | |
| carbonate) | | | | |
| Cetiol CC | 18 | 18 | 18 | 18 |
| Amphisol K | 6 | 4 | 2 | 0.6 |

TABLE 2

| | E3 | C3 |
|---|---|---|
| Water | 64.8 | 63.4 |
| Amigel | 0.3 | 0.3 |
| phenonip XB | 1 | 1 |
| Pemulen TR-2 | 0.3 | 0.3 |
| UV-Absorbing Polyester A | 15 | |
| Cetiol CC | 18 | 18 |
| Amphisol K | | 2 |
| Emulsiphos | 0.6 | |
| ¹Conventional organic | | 15 |
| sunscreen package | | |

AMIGEL is sclerotium gum, available from Alban Muller International of Hialeah, Florida.
PHENONIP XB is phenoxyethanol (and) methylparaben (and) ethylparaben (and) propylparaben, available from Clariant of Muttenz, Switzerland.
PEMULEN TR-2 is Acrylates/C₁₀₋₃₀ Alkyl Acrylate Crosspolymer, available from Noveon/Lubrizol of Wickliffe, Ohio.
CETIOL CC is Dicaprylyl Carbonate, available from Cognis, now BASF of Ludwigshafen, Germany.
AMPHISOL K is a potassium cetyl phosphate (100% anionic), available from DSM of of Heerlen, Netherlands.
Emulsiphos is potassium cetyl phosphate (60% anionic) and hydrogenated palm glycerides (40%) available from Symrise of Teterboro, New Jersey.
¹Conventional organic sunscreen package includes 5.5% homosalte, 2.8% octisalate, 2.2% octocrylene, 2.8% oxybenzone, and 1.7% avobenzone.

The UV-Absorbing Polyester A used in Example 1 was prepared by transesterification reaction of the following monomers: (1) dimerdiol, C₃₆H₇₂O, (2) di-trimethylolpropane, C₁₂H₂₆O₅, (3) dimethyladipate, and (4) benzenepropanoic acid, 3-(2h-benzotriazol-2-yl)-5-(1,1-dimethylethyl)-4-hydroxy-, methylester, C₂₀H₂₃N₃O₃. The mole ratio of the four monomers (monomer 1:monomer 2:monomer 3:monomer 4) was 2.4:3.1:4.0:8.0. The molecular weight was estimated to be about 4661 Daltons. The resulting UV-absorbing polyester was combined with a sufficient amount of dicaprylyl carbonate (CETIOL CC) to form a UV-absorbing polyester solution that was 80% by weight UV-absorbing polyester and 20% by weight dicaprylyl carbonate.

Inventive Examples E1-E3 and Comparative Examples C1-C3 were made by the following process. A water phase was prepared by adding water to a main vessel and heating to 80° C. with mixing. AMIGEL, PEMULEN TR2 and PHENONIP XB were added and mixed until dissolved. An oil phase was prepared by charging a vessel with CETIOL CC and mixing. At 60° C. UV-Absorbing Polyester A, or the conventional organic sunscreen package blend, respectively, was added. AMPHISOL K or EMULSIPHOS was added, and the mixture was heated to about 80° C. under mixing. The heated water phase was added to the oil phase with moderate shear. Moderate mixing was continued during cooling.

The MODIFIED TEP values of Inventive Examples E1-E3 and Comparative Example C1-C2 were determined using the MODIFIED TEP as described above and the results reported in Table 3.

TABLE 3

| EXAMPLE | MODOFIED TEP VALUE |
|---|---|
| E1 | 0.18 |
| E2 | 0.24 |
| E3 | 0.09 |
| C1 | 0.80 |
| C2 | 0.46 |
| C3 | 0.80 |

The results of MODIFIED TEP testing indicate that the inventive examples have very low MODIFIED TEP values, which is indicative of surprisingly low irritation. In contrast, the comparative compositions C1 and C2, containing greater than 3% anionic emulsifier, and C3, having less than 3% anionic emulsifier but containing a conventional organic sunscreen package, have much greater MODIFIED TEP values.

It is understood that while the invention has been described in conjunction with the detailed description thereof, that the foregoing description is intended to illustrate and not limit the scope of the invention.

1. A composition, comprising:
a continuous water phase,
a discontinuous oil phase substantially distributed in said water phase, said oil phase comprising a sunscreen agent consisting essentially of a UV-absorbing polyester in an amount effective to provide said composition with an SPF of about 10 or greater, which UV-absorbing polyester comprises the polymerization reaction product of monomers comprising a UV-absorbing triazole, a diester, a diol and a tetrol polyol; and
an oil-in-water emulsifier component comprising an anionic oil-in-water emulsifier in an amount of about 0.3 percent to about 3 percent by weight of said composition,
wherein said composition is substantially free of a non-polymeric UV-absorbing sunscreen agent.

2. The composition of claim 1 comprising from about 5% to about 50% by weight of said UV-absorbing polyester.

3. (canceled)

4. The composition of claim 1 comprising about 7% or more by weight of said UV-absorbing polyester.

5. The composition of claim 1 comprising about 12% or more by weight of said UV-absorbing polyester.

6. The composition of claim 1 wherein said UV-absorbing polyester has a weight average molecular weight of about 4,000 Daltons or more.

7. The composition of claim 1 wherein the UV-absorbing triazole, the diester, the diol and the tetrol polyol are reacted such that the mole fraction of UV-absorbing triazole, relative to the total number of moles of monomer used in the reaction, is from about 0.39 to about 0.60.

8. The composition of claim 7 wherein said anionic oil-in-water emulsifier is present in said composition at a concentration by weight from about 0.3% to about 2.5%.

9. The composition of claim 8 wherein said anionic oil-in-water emulsifier component comprises a potassium cetyl phosphate.

10. The composition of claim 1 wherein said anionic oil-in-water emulsifier is selected from a group consisting of alkyl, aryl or alkylaryl, or acyl-modified versions of sulfates, ether sulfates, monoglyceryl ether sulfates, sulfonates, sulfosuccinates, ether sulfosuccinates, sulfosuccinamates, amidosulfosuccinates, carboxylates, amidoethercarboxylates, succinates, sarcosinates, amino acids, taurates, sulfoacetates and phosphates.

11. The composition of claim 1 wherein said anionic oil-in-water emulsifier component comprises a phosphate ester.

12. The composition of claim 1 wherein said anionic oil-in-water emulsifier component comprises a potassium cetyl phosphate.

13. The composition of claim 1, wherein said oil-in-water emulsifier component is essentially free of non-ionic emulsifiers having an alcohol-functional group.

14. The composition of claim 1, wherein said oil-in-water emulsifier component is essentially free of cationic emulsifiers.

15. The composition of claim 1, wherein said oil-in-water emulsifier component consists essentially of said anionic emulsifier.

16. The composition of claim 1 having an SPF of about 20 or greater.
